(19) United States
(12) Patent Application Publication
(10) Pub. No.: US 2026/0083556 A1
(43) Pub. Date: Mar. 26, 2026

(54) OUTER SKIRT FOR AN EXPANDABLE PROSTHETIC HEART VALVE

(71) Applicant: Edwards Lifesciences Corporation, Irvine, CA (US)

(72) Inventor: Tamir S. Levi, Zikhron Yaakov (IL)

(73) Assignee: Edwards Lifesciences Corporation, Irvine, CA (US)

(21) Appl. No.: 19/407,084

(22) Filed: Dec. 3, 2025

Related U.S. Application Data
(63) Continuation of application No. PCT/US2024/034876, filed on Jun. 21, 2024.
(60) Provisional application No. 63/522,328, filed on Jun. 21, 2023.

(51) Int. Cl. A61F 2/24

(52) U.S. Cl. CPC A61F 2/2418; A61F 2210/0057

(57) ABSTRACT

Prosthetic heart valves are disclosed. As an example, a prosthetic heart valve comprises a frame having an inflow end and an outflow end that is expandable between a radially compressed configuration and a radially expanded configuration; and a skirt disposed around an outer surface of the frame and having an inflow portion and an outflow portion, the skirt comprising a first plurality of axially extending fibers disposed in the inflow portion and a second plurality of axially extending fibers disposed in the outflow portion, wherein each of the first plurality of fibers include two fixed ends, wherein at least one of the second plurality of fibers includes a free end and a fixed end.

### CROSS REFERENCE TO RELATED APPLICATIONS

This application is a continuation of International Application No. PCT/US2024/034876, filed Jun. 21, 2024, which claims the benefit of U.S. Provisional Patent Application No. 63/522,328, filed Jun. 21, 2023, each of which applications is incorporated herein by reference in its entirety.

### FIELD

The present disclosure relates to expandable prosthetic heart valves, including outer skirts for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Most expandable, prosthetic heart valves comprise a cylindrical metal frame or stent and prosthetic leaflets mounted inside the frame. These valves can also include one or more coverings (or skirts) spanning a circumference of the frame, on an inner or outer surface of the frame. These coverings can be configured to establish a seal with the native tissue when the prosthetic heart valve is placed at the implantation site (and thus may be referred to as sealing members). However, the native tissue (for example, at the native valve annulus or arterial wall around the native valve) can have an irregular shape while the frame of the prosthetic heart valve is generally cylindrical. As a result, gaps can be formed between the prosthetic heart valve and native heart valve annulus when the prosthetic heart valve is implanted within the native heart valve annulus, even when coverings are included on the prosthetic heart valve. Accordingly, a need exists for improved coverings or outer skirts for prosthetic heart valves.

### SUMMARY

Described herein are prosthetic heart valves, delivery apparatus, and methods for implanting prosthetic heart valves. In particular, described herein are examples of outer skirts for prosthetic heart valves and methods of making and using such outer skirts. The disclosed outer skirts, prosthetic heart valves, and methods can, for example, form a seal against native tissue upon implantation of the prosthetic heart valve, thereby reducing paravalvular leakage (PVL) past the prosthetic heart valve when expanded against the native anatomy. As such, the devices and methods disclosed herein can, among other things, overcome one or more of the deficiencies of typical prosthetic heart valves and their delivery apparatus.

A prosthetic heart valve can comprise a frame and a valve structure coupled to the frame. In addition to these components, a prosthetic heart valve can further comprise one or more of the components disclosed herein.

In some examples, a prosthetic heart valve can comprise a sealing member configured to reduce paravalvular leakage.

In some examples, a prosthetic heart valve can comprise a sealing member having axially-extending floating yarns and circumferentially-extending leno lines, wherein a first grouping of the floating yarns are woven into the leno lines and a second grouping of the floating yarns are not woven into the leno lines.

In some examples, a prosthetic heart valve can comprise a sealing member having axially-extending floating yarns and circumferentially-extending leno lines, wherein a leno line disposed at an outflow end of the sealing member is elastically deformable.

In some examples, a prosthetic heart valve can comprise a sealing member having a first grouping of floating yarns are disposed adjacent to a frame and a second grouping of floating yarns are positioned radially outward of the first grouping of floating yarns, wherein the floating yarns of the second grouping are texturized and/or have non-woven ends.

In some examples, a prosthetic heart valve comprises a frame having an inflow end and an outflow end that is expandable between a radially compressed configuration and a radially expanded configuration; and a skirt disposed around an outer surface of the frame and having an inflow portion and an outflow portion, the skirt comprising a first plurality of axially extending fibers disposed in the inflow portion and a second plurality of axially extending fibers disposed in the outflow portion, wherein each of the first plurality of fibers include two fixed ends, wherein at least one of the second plurality of fibers includes a free end and a fixed end.

In some examples, a prosthetic heart valve comprises an annular frame having an inflow end and an outflow end; and a skirt disposed around an outer surface of the frame, the skirt comprising a plurality of circumferentially-extending leno lines that are spaced axially apart from one another and a plurality of floating yarns disposed between adjacent leno lines, wherein a first grouping of the floating yarns are woven yarns that are woven into adjacent leno lines and a second grouping of the floating yarns are non-woven yarns that are not woven into at least one adjacent leno line.

In some examples, a prosthetic heart valve comprises a frame; and a skirt coupled to the frame, the skirt comprising circumferential leno lines and axial yarns extending between the leno lines, wherein a first grouping of the axial yarns are woven into the leno lines, wherein the first grouping of the axial yarns defines a barrier between a second grouping of the axial yarns and the frame.

In some examples, a prosthetic heart valve comprises a frame comprising a plurality of interconnected struts defining a plurality of circumferentially extending rows of cells including a row of outflow cells disposed at the outflow end of the frame, wherein the row of outflow cells are longer in an axial direction relative to remaining rows of cells of the plurality of rows of cells; and an outer skirt coupled to the frame, the skirt comprising a plurality of circumferential yarns and a plurality of axial yarns extending between the circumferential yarns, wherein a first circumferential yarn is elastically deformable and extends across at least a portion of each outflow cell of the row of outflow cells.

In some examples, a prosthetic heart valve comprises a frame having an inflow end and an outflow end; and a skirt coupled to the frame, the skirt comprising a plurality of circumferential yarns and a plurality of axial yarns extending between the circumferential yarns, wherein the plurality of circumferential yarns include a first circumferential yarn that is positioned towards the outflow end and a second circumferential yarn that is positioned closer to the inflow end than the first circumferential yarn, wherein a first grouping of the axial yarns include a first woven region that is woven with the first circumferential yarn and a second woven region that is woven with the second circumferential yarn, and wherein a second grouping of the axial yarns include a woven region that is woven with the second circumferential yarn and a free region positioned adjacent to the first circumferential yarn that is not-woven into the first circumferential yarn and free to move radially outwards relative to the first circumferential yarn.

In some examples, a prosthetic heart valve comprises one or more of the components recited in Examples 1-54 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2 is a side view of a frame of the prosthetic heart valve of FIG. 1.

FIG. 3 is a side view of a portion of the frame of FIG. 2, showing the portion of the frame in a straightened (non-annular) state.

FIG. 4 is a side view of an exemplary delivery apparatus configured to deliver and implant a radially expandable prosthetic heart valve at an implantation site.

FIG. 5 is a side view of a portion of an outer skirt, according to one example, disposed around an outer surface of a portion of an exemplary frame of a prosthetic heart valve.

FIGS. 6A-6B illustrate detailed views of the outer skirt of FIG. 5 illustrating yarns of the outer skirt.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

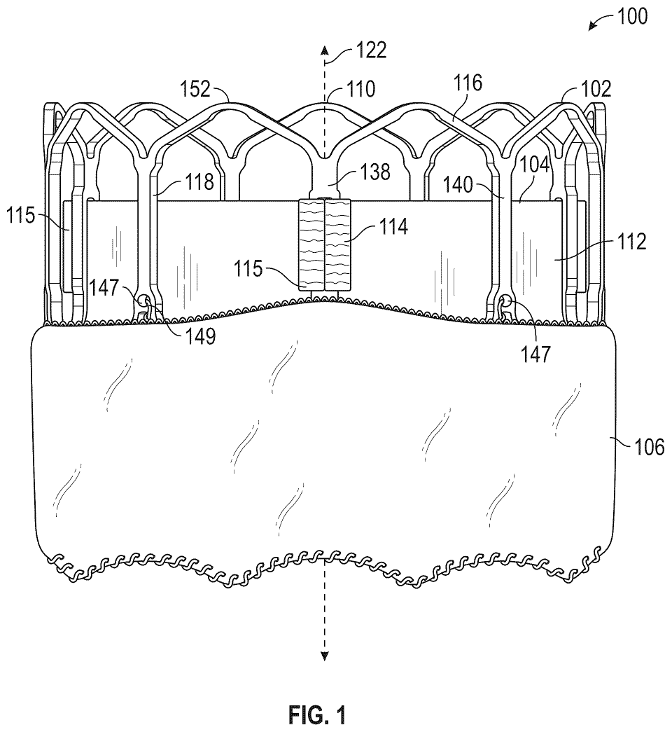

As introduced above, most prosthetic heart valve can include an outer skirt disposed around an outer surface of an annular frame of the prosthetic heart valve. The outer skirt can be configured to form a seal against native tissue upon implantation of the prosthetic heart valve, thereby reducing paravalvular leakage (PVL) past the prosthetic heart valve when expanded against the native anatomy. FIGS. 1-3 illustrate an exemplary prosthetic device (for example, prosthetic heart valve) comprising a frame, leaflets secured on an inside of the frame, and an outer skirt disposed around an outer surface of the frame. The prosthetic device can be advanced through a patient's vasculature, such as to a native heart valve, by a delivery apparatus, such as the exemplary delivery apparatus shown in FIG. 4.

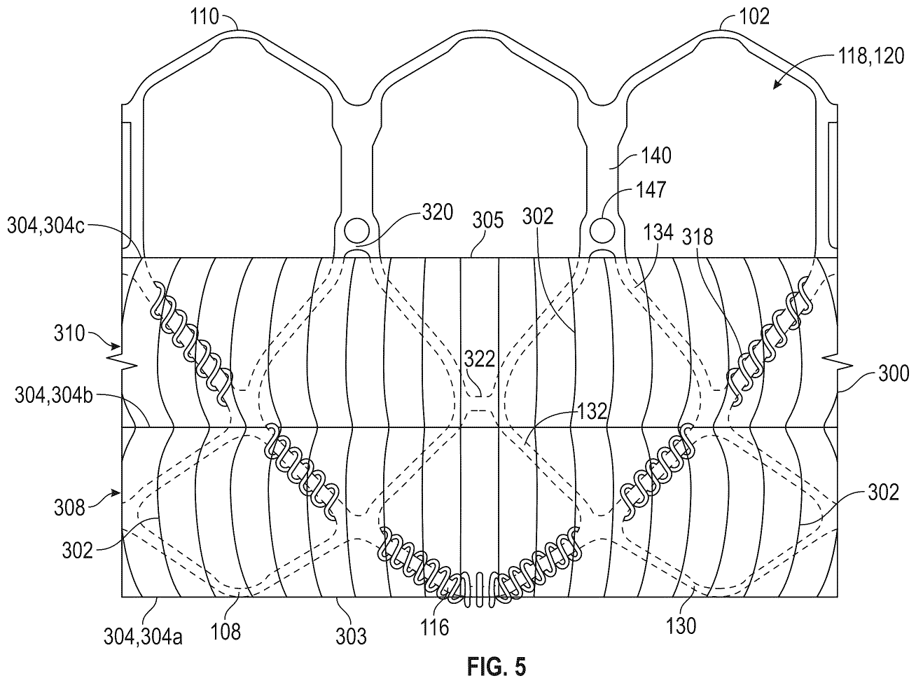

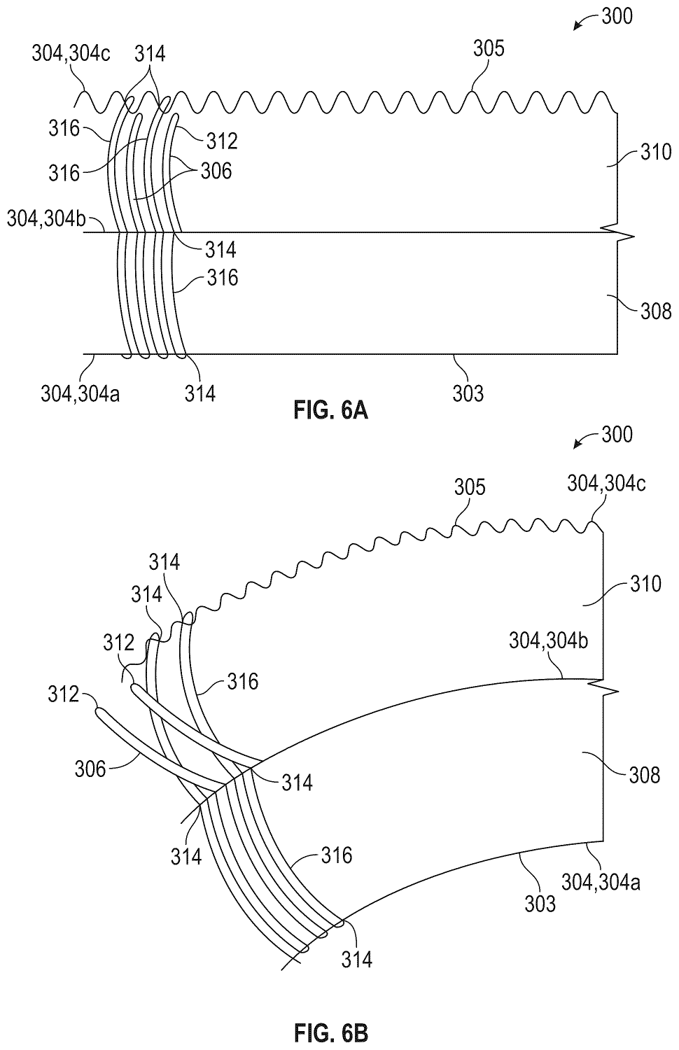

In some examples, an outer skirt for a prosthetic heart valve, such as the outer skirts depicted in FIGS. 5-6B, can be configured with portions that extend radially outward from the frame of the prosthetic heart valve (toward the surrounding native anatomy) to increase PVL sealing against the native anatomy. For example, the outer skirts shown in FIGS. 5-6B can comprise an arrangement of floating yarns or fibers extending between spaced apart leno weaves (also referred to herein as leno lines). It should be understood that any reference herein to a “fiber” or “fibers” is also intended to encompass a “yarn” or “yarns”. For example, a skirt constructed from floating individual or discrete fibers can also be constructed from floating yarns wherein each yarn comprises a collection of a plurality of fibers (for example, a collection of twisted fibers). In some examples, one or more of the floating fibers can include a portion (for example, an end, a loop, etc.) that is free or not woven into an adjacent leno line, such that the free portion of the floating fiber is free to extend radially outward from the frame while being prevented from extending radially inward (for example, into the frame) by floating fibers that are fully woven into the leno lines. As a result, the PVL sealing of the prosthetic heart valve can be increased and a separation between the outer skirt and leaflets disposed within the frame can be maintained, thus preventing unnecessary and/or unwanted contact between the leaflets and the outer skirt.

### Examples of the Disclosed Technology

FIG. 1 shows a prosthetic heart valve 100 (prosthetic valve), according to one example. Any of the prosthetic valves disclosed herein are adapted to be implanted in the native aortic annulus, although in some examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in International Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic heart valve 100 can include a stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102.

The valvular structure 104 can comprise a plurality of leaflets 112 (for example, three leaflets, as shown in FIG. 1), collectively forming a leaflet structure, which can be arranged to collapse in a tricuspid arrangement. The leaflets 112 can be secured to one another at their adjacent sides (for example, commissure tabs) to form commissures 114 of the valvular structure 104. For example, each leaflet 112 can comprise opposing commissure tabs disposed on opposite sides of the leaflet 112 and a cusp edge portion extending between the opposing commissure tabs. The cusp edge portion of the leaflets 112 can have an undulating, curved scalloped shape, and can be secured directly to the frame 102 (for example, by sutures). However, in alternate examples, the cusp edge portion of the leaflets 112 can be secured to an inner skirt which is then secured to the frame 102. In some examples, the leaflets 112 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

In some examples, the outer skirt 106 can be an annular skirt. In some instances, the outer skirt 106 can comprise one or more skirt portions that are connected together and/or individually connected to the frame 102. The outer skirt 106 can comprise a fabric or polymeric material, such as ePTFE, PTFE, PET, TPU, UHMWPE, PEEK, PE, etc. In some instances, instead of having a relatively straight upper edge portion, as shown in FIG. 1, the outer skirt 106 can have an undulating upper edge portion that extends along and is secured to the angled struts 134. Examples of such outer skirts, as well as various other outer skirts, that can be used with the frame 102 can be found in U.S. Provisional Application No. 63/366,599, filed Jun. 17, 2022, which is incorporated by reference herein.

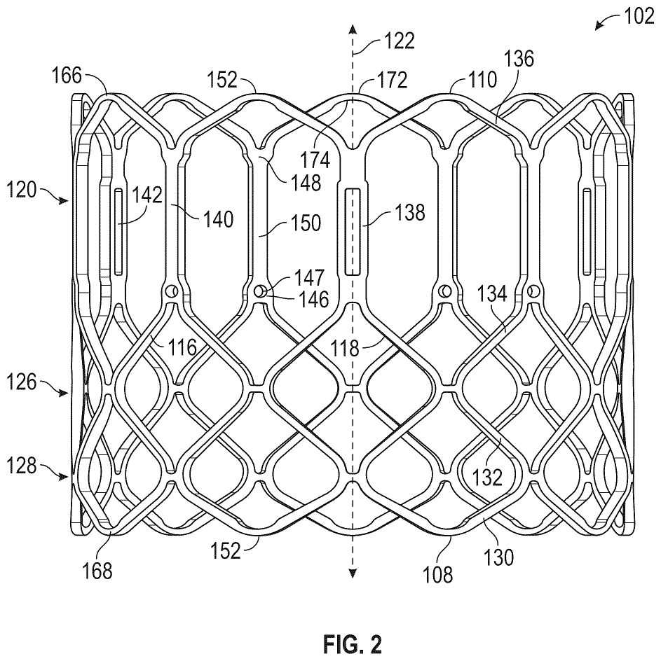

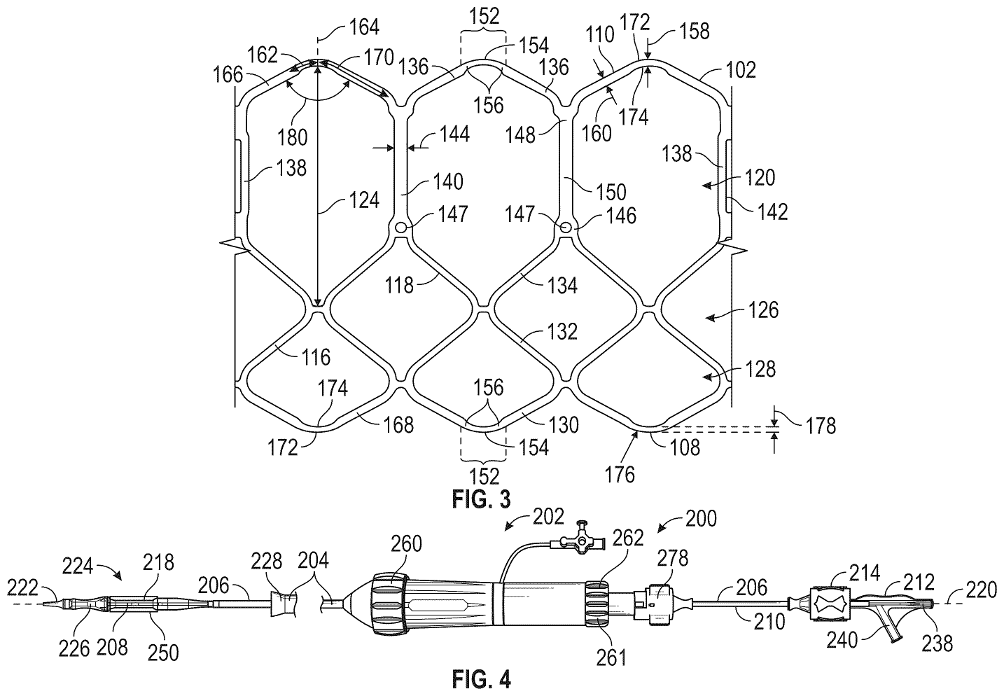

The frame 102 can be radially compressible and expandable between a radially compressed configuration and a radially expanded configuration (the expanded configuration is shown in FIG. 1). The frame 102 is shown alone in FIG. 2 and a portion of the frame 102 in a straightened (non-annular) configuration is shown in FIG. 3.

The frame 102 can be made of any of various suitable plastically-expandable materials (for example, stainless steel, etc.) or self-expanding materials (for example, Nitinol) as known in the art. When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100 can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIGS. 2 and 3, the frame 102 can comprise a plurality of interconnected struts 116 which form multiple rows of open cells 118 between the outflow end 110 and the inflow end 108 of the frame 102. In some examples, as shown in FIGS. 2 and 3, the frame 102 can comprise three rows of cells 118 with a first (upper in the orientation shown in FIGS. 2 and 3) row of cells 120 disposed at the outflow end 110. The first row of cells 120 comprises cells 118 that are elongated in an axial direction (relative to a central longitudinal axis 122 of the frame 102), as compared to cells 118 in the remaining rows of cells. For example, the cells 118 of the first row of cells 120 can have a longer axial length 124 (FIG. 3) than cells 118 in the remaining rows of cells, which can include a second row of cells 126 and a third row of cells 128, the third row of cells 128 disposed at the inflow end 108 and the second row of cells 126 disposed between the first row of cells 120 and the third row of cells 128.

In some examples, as shown in FIG. 2, each row of cells comprises nine cells 118. Thus, in such examples, the frame 102 can be referred to as a nine-cell frame.

In alternate examples, the frame 102 can comprise more than three rows of cells (for example, four or five) and/or more or less than nine cells per row. In some examples, the cells 118 in the first row of cells 120 may not be elongated compared to cells 118 in the remaining rows of cells of the frame 102 (the second row of cells 126 and the third row of cells 128).

The interconnected struts 116 can include a plurality of angled struts 130, 132, 134, and 136 arranged in a plurality of rows of circumferentially extending rows of angled struts, with the rows being arrayed along the length of the frame 102 between the outflow end 110 and the inflow end 108. For example, the frame 102 can comprise a first row of angled struts 130 arranged end-to-end and extending circumferentially at the inflow end 108 of the frame; a second row of circumferentially extending, angled struts 132; a third row of circumferentially extending, angled struts 134; and a fourth row of circumferentially extending, angled struts 136 at the outflow end 110 of the frame 102. The fourth row of angled struts 136 can be connected to the third row of angled struts 134 by a plurality of axially extending window struts 138 (or window strut portions) and a plurality of axial (for example, axially extending) struts 140. The axially extending window struts 138 (which can also be referred to as axial struts that include a commissure window) define commissure windows (for example, open windows) 142 that are spaced apart from one another around the frame 102, in a circumferential direction, and which are adapted to receive a pair of commissure tabs of a pair of adjacent leaflets 112 arranged into a commissure (for example, commissure 114 shown in FIG. 1). In some examples, the commissure windows 142 and/or the axially extending window struts 138 defining the commissure windows 142 can be referred to herein as commissure features or commissure supports, each commissure feature or support configured to receive and/or be secured to a pair of commissure tabs of a pair of adjacent leaflets.

One or more (for example, two, as shown in FIGS. 2 and 3) axial struts 140 can be positioned between, in the circumferential direction, two commissure windows 142 formed by the window struts 138. Since the frame 102 can include fewer cells per row (for example, nine) and fewer axial struts 140 between each commissure window 142, as compared to some more traditional prosthetic heart valves, each cell 118 can have an increased width (in the circumferential direction), thereby providing a larger opening for blood flow and/or coronary access.

Each axial strut 140 and each window strut 138 extends from a location defined by the convergence of the lower ends (for example, ends arranged inward of and farthest away from the outflow end 110) of two angled struts 136 (which can also be referred to as an upper strut junction or upper elongated strut junction) to another location defined by the convergence of the upper ends (for example, ends arranged closer to the outflow end 110) of two angled struts 134 (which can also be referred to as a lower strut junction or lower elongate strut junction). Each axial strut 140 and each window strut 138 forms an axial side of two adjacent cells of the first row of cells 120.

In some examples, as shown in FIG. 3, each axial strut 140 can have a width 144 (FIG. 3) that is larger than a width of the angled struts 130, 132, 134, and 136. As used herein, a “width” of a strut is measured between opposing locations on opposing surfaces of a strut that extend between the radially facing inner and outer surfaces of the strut (relative to the central longitudinal axis 122 of the frame 102). A “thickness” of a strut is measured between opposing locations on the radially facing inner and outer surfaces of a strut and is perpendicular to the width of the strut. In some examples, the width 144 of the axial struts 140 is 50-200%, 75-150%, or at least 100% larger than (for example, double) the width of the angled struts of the frame 102.

By providing the axial struts 140 with the width 144 that is greater than the width of other, angled struts of the frame 102, a larger contact area is provided for when the leaflets 112 contact the wider axial struts 140 during systole, thereby distributing the stress and reducing the extent to which the leaflets 112 may fold over the axial struts 140, radially outward through the cells 118. As a result, a long-term durability of the leaflets 112 can be increased.

Since the cells 118 of the frame 102 can have a relatively large width compared to alternate prosthetic valves that have more than nine cells per row (as introduced above), the wider axial struts 140 can be more easily incorporated into the frame 102, without sacrificing open space for blood flow and/or coronary access.

Commissure tabs 115 of adjacent leaflets 112 can be secured together to form commissures 114 (FIG. 1). Each commissure 114 of the prosthetic heart valve 100 comprises two commissure tabs 115 paired together, one from each of two adjacent leaflets 112, and extending through a commissure window 142 of the frame 102. Each commissure 114 can be secured to the window struts 138 forming the commissure window 142.

The cusp edge portion (for example, scallop edge) of each leaflet 112 can be secured to the frame 102 via one or more fasteners (for example, sutures). In some examples, the cusp edge portion of each leaflet 112 can be secured directly to the struts of the frame 102 (for example, angled struts 130, 132, and 134). For example, the cusp edge portions of the leaflets 112 can be sutured to the angled struts 130, 132, and 134 that generally follow the contour of the cusp edge portions of the leaflets 112.

In some examples, the cusp edge portion of the leaflets 112 can be secured to an inner skirt and the inner skirt can then be secured directly to the frame 102.

Various methods for securing the leaflets 112 to a frame, such as the frame 102, are disclosed in U.S. provisional patent applications 63/278,922, filed Nov. 12, 2021, and 63/300,302, filed Jan. 18, 2022, both of which are incorporated by reference herein.

As shown in FIGS. 2 and 3, in some examples, one or more of or each of the axial struts 140 can comprise an inflow end portion 146 (for example, an end portion that is closest to the inflow end 108) and an outflow end portion 148 that are widened relative to a middle portion 150 of the axial strut 140 (which can be defined by the width 144). In some instances, the inflow end portion 146 of the axial strut 140 can comprise an aperture 147. The apertures 147 can be configured to receive fasteners (for example, sutures) for attaching soft components of the prosthetic heart valve 100 to the frame 102. For example, in some instances, the outer skirt 106 can be positioned around the outer surface of the frame 102 and an upper or outflow edge portion of the outer skirt 106 can be secured to the apertures 147 by fasteners 149 (for example, sutures), as shown in FIG. 1.

The frame 102 can further comprise a plurality of apex regions 152 formed at the inflow end 108 and the outflow end 110, each apex region 152 extending and forming a junction between two angled struts 130 at the inflow end 108 or two angled struts 136 at the outflow end 110. As such, the apex regions 152 are spaced apart from one another, in a circumferential direction at the inflow end 108 and the outflow end 110.

Each apex region 152 can comprise an apex 154 (the highest or most outward extending, in an axial direction, point) and two thinned (or narrowed) strut portions 156, one thinned strut portion 156 extending from either side of the apex 154 to a corresponding, wider, angled strut 136 (at the outflow end 110) or angled strut 130 (at the inflow end 108) (FIG. 3). In this way, each of the apex regions 152 at the outflow end 110 can form a narrowed transition region between and relative to the two angled struts 136 extending from the corresponding apex region 152 and each of the apex regions 152 at the inflow end 108 can form a narrowed transition region between and relative to the two angled struts 130 extending from the corresponding apex region 152.

The thinned strut portions 156 of the apex regions 152 can have a width 158 that is smaller than a width 160 of the angled struts 130 or 136 (FIG. 3). In some examples, the width 158 can be a uniform width (for example, along an entire length of the strut portion 156). In some examples, the width 158 of the thinned strut portions 156 can be from about 0.06-0.15 mm smaller than the width 160 of the angled struts 130 and/or 136.

The thinned strut portions 156 of the apex regions 152 can have a first length 162 (FIG. 3). In some examples, the first length 162 is in a range of 0.8-1.4 mm, 0.9-1.2 mm, 0.95-1.05 mm, or about 1.0 mm (for example, ±0.03 mm). In alternate examples, the first length 162 is in a range of 0.3-0.7 mm, 0.4-0.6 mm, 0.45-0.55 mm, or about 0.5 mm (for example, +0.03 mm).

Thus, each outflow apex region 152 can include two thinned strut portions 156 having the first length 162, each extending from the apex 154, outward relative to a central longitudinal axis 164 of the cells 118. Thus, a total length of the apex region 152 can be two times the first length 162.

Each apex region 152 and two corresponding angled struts 136 at the outflow end 110 can form an outflow strut 166 and each apex region 152 and two corresponding angled struts 130 at the inflow end 108 can form an inflow strut 168.

Each outflow strut 166 and inflow strut 168 can have a length that includes an apex region 152 and the two angled struts 136 or 130 (or strut portions), respectively, on either side of the apex region 152. One half the total length of each outflow strut 166 and inflow strut 168 is shown in FIG. 3 as length 170, which extends from an end of one angled strut 136 or 130 to the central longitudinal axis 164. Thus, the length of each outflow strut 166 and inflow strut 168 is two times length 170. In some examples, the length 170 for half of each inflow strut 168 can be different than the length 170 for half of each outflow strut 166.

In some instances, the length of each thinned strut portion 156 can be at least 25% of the length 170 of the corresponding half outflow strut 166 or inflow strut 168. Said another way, the length of each apex region 152 (a total length being two times the first length 162) can be at least 25% of the total length (two times length 170) of the outflow strut 166 or inflow strut 168. In some examples, the length of each apex region 152 can be more than 25% of the total length of the corresponding outflow strut 166 or inflow strut 168, such as 25-35%.

In some examples, each apex region 152 can comprise a curved, axially facing outer surface 172 and an arcuate or curved, axially facing inner depression 174 which forms the thinned strut portions 156. For example, the curved inner depression 174 can depress toward the curved outer surface 172 from an inner surface of the angled strut portions 156, thereby forming the smaller width thinned strut portions 156. Thus, the curved inner depressions 174 can be formed on a cell side of the apex region 152 (for example, as opposed to the outside of the apex region 152).

In some examples, the curved outer surface 172 of each apex region 152 can form a single, continuous curve from one angled strut portion 156 on a first side of the apex region 152 to another angled strut portion 156 on an opposite, second side of the apex region 152.

Each apex region 152 can have a radius of curvature 176, along the curved outer surface 172 (for example, in some instances, along an entirety or an entire length of the curved outer surface 172) (FIG. 3). In some instances, the radius of curvature 176 at the apex 154 and/or along the entire curved outer surface 172 of the apex region 152 can be greater than 1 mm. In some instances, the radius of curvature 176 can be in a range of 1-20 mm, 3-16 mm, or 8-14 mm. In some instances, the radius of curvature 176 can be greater than 10 mm. The radius of curvature 176 can be dependent on (and thus change due to changes in) the width 158 (for example, the amount of reduction in width from the angled struts 130 or 136) and the first length 162 of the thinned strut portions 156.

Further, a height (an axial height) 178 of the apex regions 152, which can be defined in the axial direction from an outer surface of the two angled struts 130 or 136 to the curved outer surface 172 of the apex region 152 at the apex 414, can be the width 158 of the thinned strut portions 156 (FIG. 3). In this way, the height 178 of the apex regions 152 can be relatively small and not add much to the overall axial height of the radially expanded frame 102. Thus, the leaflets 112 secured to the frame 102 (FIG. 1) can be disposed close to the inflow end 108, thereby leaving a larger open space at the outflow end 110 of the frame 102 that is not blocked by the leaflets 112.

In some examples, each of the apex region 152 can form an angle 180 between the two angled struts 130 or 136 extending from either side of the corresponding apex region 152 (FIG. 3). In some instances, the angle 180 can be in a range of 120 (not inclusive) to 140 degrees (for example, such that the angle 180 is greater than 120 degrees and less than or equal to 140 degrees).

Additional details and examples of frames for prosthetic heart valves that include apex regions can be found in International Application No. PCT/US2022/025687, filed Apr. 21, 2022, which is incorporated by reference herein.

FIG. 4 shows a delivery apparatus 200, according to an example, that can be used to implant an expandable prosthetic heart valve (for example, the prosthetic heart valve 100 of FIG. 1 and/or any of the other prosthetic heart valves described herein). In some examples, the delivery apparatus 200 is specifically adapted for use in introducing a prosthetic valve into a heart.

The delivery apparatus 200 in the illustrated example of FIG. 4 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (for example, move) longitudinally, along a central longitudinal axis 220 of the delivery apparatus 200, relative to one another to facilitate delivery and positioning of a prosthetic valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (for example, inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222 (as shown in FIG. 4), or to an alternate component at the distal end of the delivery apparatus 200 (for example, a distal shoulder). An intermediate portion of the balloon 218 can overlay a valve mounting portion 224 of a distal end portion of the delivery apparatus 200 and a distal end portion of the balloon 218 can overly a distal shoulder 226 of the delivery apparatus 200. The valve mounting portion 224 and the intermediate portion of the balloon 218 can be configured to receive a prosthetic heart valve in a radially compressed state. For example, as shown schematically in FIG. 4, a prosthetic heart valve 250 (which can be one of the prosthetic valves described herein) can be mounted around the balloon 218, at the valve mounting portion 224 of the delivery apparatus 200.

The balloon shoulder assembly, including the distal shoulder 226, is configured to maintain the prosthetic heart valve 250 (or other medical device) at a fixed position on the balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic valve 250 is mounted in the radially compressed state on the valve mounting portion 224 (as shown in FIG. 4) and during delivery of the prosthetic valve to the target implantation site. As such, the distal tip portion 228 can be configured to resist movement of the prosthetic valve 250 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (for example, for fine positioning at the implantation site). Further details on the delivery apparatus 200 can be found in International Application No. PCT/US2021/047056, which is incorporated by reference herein.

FIG. 5 shows one example of an outer skirt 300 for a prosthetic device, such as the prosthetic heart valve 100 of FIG. 1, in a flattened configuration. As described in further detail below, the outer skirt 300 is configured to increase PVL leakage sealing with the native anatomy once the prosthetic device has been radially expanded within the native anatomy (such as at a native heart valve annulus). The outer skirt 300 can be used in a mechanically expandable prosthetic valve, a balloon-expandable prosthetic valve (for example, prosthetic heart valve 100 of FIG. 1), and/or a self-expandable prosthetic valve. In some examples, as shown, the outer skirt 300 can be used in lieu of the outer skirt 106 on prosthetic valve 100. Additional details on balloon expandable prosthetic valves can be found in International Application No. PCT/US2022/025687, filed Apr. 21, 2022, as already incorporated by reference herein. Additional details on a mechanically expandable prosthetic valve can be found in International Application PCT/US2021/052745, filed Sep. 30, 2021, which is incorporated by reference herein. Additional details on a self-expanding prosthetic valve can be found in U.S. Pat. No. 8,652,202, which is incorporated by reference herein.

The outer skirt 300 can comprise a fabric body formed from a plurality of strands or yarns that are woven, knitted, or otherwise secured together. In some examples, as depicted, the outer skirt 300 can include a plurality of yarns 302 extending in an axial direction (up-and-down or vertically in FIG. 5) (“axial yarns 302” or “floating yarns 302”) and a plurality of yarns 304 extending in a circumferential direction (side-to-side or horizontally in FIG. 5) (“circumferential yarns 304”), for example, as coupled to a frame 102. While the floating yarns 302 are illustrated as oriented in an axial direction extending between an inflow end 303 and an outflow end 305 of the skirt 300 (for example, perpendicular to the circumferential yarns 304), in some examples, the floating yarns 302 can be angled relative to the circumferential yarns 304.

The outer skirt 300 can be segmented into multiple, axial portions or segments by one or more of the circumferential yarns 304. For example, as depicted in FIG. 5, the outer skirt 300 can include an inflow portion 308 and an outflow portion 310. As shown, the inflow portion 308 is disposed at or adjacent to the inflow end 108 of the frame 102 and the outflow portion 310 is disposed towards or adjacent to the outflow end 110 of the frame 102. The floating yarns 302 can be included in both the inflow portion 308 and the outflow portion 310.

In some examples, one or more of the circumferential yarns 304 can be leno yarns or fibers that are woven in a leno weave pattern with at least a portion of the floating yarns 302, such as disclosed in U.S. Patent Publication 2019/0374337, as already incorporated by reference above. Accordingly, in some examples, the circumferential yarns 304 can also be referred to as circumferentially-extending, floating leno lines 304 (or woven portions). In the illustrated example, the outer skirt 300 comprises three leno lines 304 that are spaced axially apart from one another, with a first (or inflow) leno line 304a disposed at the inflow end 303 of the skirt 300, a second (or intermediate leno line 304b disposed at an intermediate location of the skirt 300, and a third (or outflow) leno line 304c disposed at the outflow end 305 of the skirt 300. In the FIG. 5, each leno line 304a, 304b, 304c is represented by a single yarn or fiber 304. However, it should be understood that each leno line 304a, 304b, 304c can comprise multiple leno yarns 304 that are interwoven with each other and the floating yarns 302.

The leno lines 304 divide the outer skirt 300 into the inflow portion 308 and the outflow portion 310. Specifically, the inflow portion 308 extends axially between the inflow leno line 304a and the intermediate leno line 304b. The outflow portion 310 extends axially between the intermediate leno line 304b and the outflow leno line 304c. While three leno lines 304 are shown in FIG. 5, in some examples, an outer skirt can include additional intermediate leno lines, similar to intermediate leno line 304b.

While two portions (i.e., inflow portion 308, outflow portion 310) and three leno lines (i.e., leno line 304a, leno line 304b, leno line 304c) are shown in FIG. 5, the outer skirt 300 can be segmented into a different number of portions, for example, by a different number of leno lines 304 in some examples. For instance, an outer skirt can include more than three leno lines and include one or more intermediate portions disposed axially between the inflow portion 308 and the outflow portion 310. Although not shown, in examples having intermediate portions, the intermediate portions can have an arrangement of yarns similar to the inflow portion 308, as described in more detail below. In some examples, not shown, the outer skirt 300 can include one portion, rather than being segmented into multiple portions, such that the outer skirt 300 is segmented by two leno lines (for example, one portion extending between leno line 304a and leno line 304c.

FIGS. 6A-6B depict a portion of the outer skirt 300 with some of the floating yarns 302 illustrated in greater detail. The floating yarns 302 are illustrated as one continuous yarn that is woven into one or more of the leno lines 304. In some examples, the outer skirt 300 includes multiple, distinct floating yarns 302 that are woven into one or more of the leno lines 304. In some instances, a floating yarn 302 can refer to a distinct yarn or a portion of a yarn that extends from a first leno line 304 to a second leno line 304. As shown in FIGS. 6A-6B, some of the floating yarns 302 are woven into all of the leno lines 304 and some of the floating yarns 302 are not woven into all of the leno lines 304. As used herein, a “catch” region of a floating yarn 302 refers to a region of a floating yarn 302 that is at a leno line 304 and is fixed or woven into the corresponding leno line 304. A “no catch” region of a floating yarn 302 refers to a region of an axial yarn (for example, an end, a loop, a portion, etc.) that is at or adjacent to the leno line 304 and is free or not woven into the corresponding leno line 304. The “no catch” region of a floating yarn 302 is free to extend radially outwards and away from the skirt 300. In some examples, “no catch” regions of floating yarns 302 can increase the surface area of the outer skirt 300 that contacts native anatomy (for example, when the prosthetic heart valve 100 is radially expanded) and increase PVL sealing.

Each floating yarn 302 can have a “catch” or “no catch” region that corresponds to the region of the floating yarn 302 that is at or adjacent to each of the leno lines 304. For example, floating yarns 302 disposed in the inflow portion 308 can have “catch” or “no catch” regions located at or adjacent to the inflow leno line 304a and the intermediate leno line 304b, while floating yarns 302 disposed in the outflow portion 310 can have “catch” or “no “catch” regions located at or adjacent to the intermediate leno line 304b and the outflow leno line 304c. In some examples, a “catch” region can also be referred to as a fixed or woven end or loop of a floating yarn 302 and a “no catch” region can also be referred to as a free or non-woven end or loop of a floating yarn 302.

A first plurality of the floating yarns 302 are non-woven yarns 306 having at least one “no catch” region 312 that is free or not woven into one of the leno lines 304 and at least one “catch” region 314 that is fixed or woven into one of the leno lines 304. As shown in FIG. 6B, the non-woven yarns 306 can extend radially outward from the leno lines 304. The non-woven yarns 306 can increase the texture of the outer surface of the outer skirt 300 and can be configured to increase tissue ingrowth following implantation of a prosthetic device including the outer skirt 300.

The non-woven yarns 306 can be disposed in the outflow portion 310, and in some examples, as shown in FIGS. 6A-6B, only in the outflow portion 310. A non-woven yarn 306 can have a “catch” region 314 at an inflow end of the non-woven yarn 306 (for example, positioned towards the inflow end 303 of the skirt 300) and have a “no catch” region 312 at an outflow end of the non-woven yarn 306 (for example, positioned towards the outflow end 305 of the skirt 300). For example, the non-woven yarns 306 have “catch” regions 314 at the intermediate leno line 304b and have “no catch” regions 312 adjacent to the outflow leno line 304c. Although not shown, in some examples, one or more of the non-woven yarns 306 can instead have “catch” regions 314 at the outflow leno line 304c and “no catch” regions 312 adjacent to the intermediate leno line 304b.

Non-woven yarns 306 can be disposed in any portion of the outer skirt 300, including the inflow portion 308 and/or the outflow portion 310. For example, although not shown, an outer skirt can include non-woven yarns 306 disposed in an inflow portion 308 and the non-woven yarns 306 can have “no catch” regions 312 at or adjacent to the inflow leno line 304a and/or the intermediate leno line 304b. Further, although not shown, in some examples, a non-woven yarn 306 can have “catch” regions 314 at both of the inflow and outflow leno lines 304a, 304c and have a “no catch” region 312 at the intermediate leno line 304b, such that the non-woven yarn 306 is free or non-woven between the inflow and outflow leno lines 304a, 304c. As another example, a non-woven yarn 306 can have one “catch” region 314 at either the inflow or outflow leno lines 304a, 304c and the non-woven yarn 306 can be otherwise free or non-woven in an axial direction from the inflow or outflow leno lines 304a, 304c (for example, a non-woven yarn 306 that includes a “catch” region 314 at the inflow leno line 304a and “no catch” regions 312 at the intermediate and outflow leno lines 304b, 304c, etc.).

A second plurality of the floating yarns 302 are woven yarns 316 having “catch” regions 314 that are fixed or woven into the leno lines 304. Specifically, the woven yarns 316 do not include any “no catch” regions 312, such that the woven yarns 316 are fully woven into the leno lines 304 (for example, at each end). In some examples, as shown in FIGS. 6A-6B, some of the floating yarns 302 disposed in the outflow portion 310 and all of the floating yarns 302 disposed in the inflow portion 308 are woven yarns 316. The woven yarns 316 disposed in the inflow portion 308 have “catch” regions 314 that are woven into the inflow leno line 304a and the intermediate leno line 304b. The woven yarns 316 disposed in the outflow portion 310 have “catch” regions 314 that are woven into the intermediate leno line 304b and the outflow leno line 304c.

In some examples, it can be beneficial for the “catch” regions 314 of the non-woven yarns 306 to be oriented closer to a distal end of the outer skirt 300 (for example, inflow end 303) than the “no-catch” regions 316. In this example, the distal end of the outer skirt 300 refers to the end of the outer skirt 300 that is positioned towards a distal end of a delivery apparatus, such as when the prosthetic heart valve 100 crimped on and/or retained by the delivery apparatus 200. For example, when the prosthetic valve 100 is mounted on the delivery apparatus for retrograde delivery to the native aortic valve (through the aorta), the inflow end 303 of the skirt is distal to the outflow end 305. In this example, the “no-catch” regions 316 of the non-woven yarns 306 can be disposed at the outflow leno line 304c and the “catch regions” 314 can be disposed at the leno lines 304a, 304b. This configuration can allow the prosthetic heart valve 100 (and the outer skirt 300) (while crimped on the delivery apparatus) to be advanced through an introducer sheath in a smooth manner, for example, without the non-woven yarns 306 resisting such advancement or being pulled out of alignment from contact with the inner surface of the introducer sheath. In other words, this configuration can allow the non-woven yarns 306 to be “combed” or pushed towards adjacent yarns of the outer skirt 300 as the prosthetic heart valve 100 is advanced through the introducer sheath, rather than the non-woven yarns 306 of the outer skirt 300 being back-combed radially away from the outer skirt 300 and resisting such advancement.

In some examples, as shown in FIGS. 6A-6B, the non-woven yarns 306 and the woven yarns 316 are arranged in an alternating pattern (for example, in a circumferential direction), such that every other floating yarn 302 is a non-woven yarn 306. In some examples, not shown, the non-woven yarns 306 and the woven yarns 316 can be arranged in other configurations, including other repeating patterns, non-uniform or random patterns, etc.

In some configurations, at least some of the floating yarns 302 are texturized or textured, such that the floating yarns 302 have an increased surface area. For example, at least some of the floating yarns 302 can comprise texturized strands (for example, texturized yarns, etc.) in which the constituent fibers of the strands/yarns have been bulked by, for example, being twisted, heat set, and untwisted such that the fibers retain their deformed, twisted shape in a relaxed, non-stretched configuration. The texturized strands/yarns can also be texturized by crimping, coiling, etc. When the texturized strands/yarns are in a relaxed, non-tensioned state, the texturized strands/yarns can be loosely packed and can provide compressible volume or bulk to the fabric, as well as a plush surface. In some instances, the texturized strands/yarns can promote tissue ingrowth and PVL sealing against the native tissue. Further details on texturized strands/yarns are disclosed in U.S. Patent Publication 2019/0374337, as already incorporated by reference herein, and International Patent Publication WO 2021/202636, which is incorporated by reference herein.

In some examples, the outer skirt 300 can have floating yarns 302 that are texturized and floating yarns 302 that are non-texturized (for example, smooth straight fibers having closely packed, parallel positioning, etc.). The texturized yarns can be disposed in the inflow portion 308 and/or the outflow portion 310 and can comprise at least some of the non-woven yarns 306 and/or at least some of the woven yarns 316. In one non-limiting example, the non-woven yarns 306 disposed in the outflow portion 310 are texturized, and the remaining yarns 302 (for example, the woven yarns 316 disposed in the inflow portion 308, the woven yarns 316 disposed in the outflow portion 310) can either be texturized or non-texturized. In this example, the texturized, non-woven yarns 306 further increase the surface area of the outer skirt 300 to promote tissue ingrowth and PVL sealing, as the texturized, non-woven yarns 306 extend radially away from the outer skirt 300 and have a textured configuration to engage with the native anatomy. In some examples, different ones of the floating yarns 302 can be texturized (for example, some, none, or all of the floating yarns 302, etc.).

As described below, the outer skirt 300 can be configured to prevent portions of the outer skirt 300 from extending into and/or loosely dangling across cells 118 of the frame 102 (for example, outflow cells 120), for example, to maintain separation between the leaflets 112 and the outer skirt 300.

Specifically, in some examples, one or more of the circumferential yarns 304 (for example, one or more yarns of leno line 304a, leno line 304b, and/or leno line 304c) can comprise elastically deformable or elastic yarns. FIGS. 6A-6B illustrate leno line 304c as an elastically deformable yarn, with leno line 304c shown as an undulating line. The elastic circumferential yarn(s) 304 can comprise an elastic material and/or be configured such that the leno line 304 elastically deforms, for example, when the prosthetic heart valve 100 is radially expanded from a radially compressed configuration to a fully expanded configuration. This can keep the outer skirt 300 relatively taut around the frame 102, regardless of the amount of radial expansion of the frame 102. As such, separation can be maintained between the outer skirt 300 and the interior of the frame 102, for example, when the prosthetic heart valve 100 is radially compressed, partially expanded, fully expanded, etc.

In some instances, as shown in FIG. 5, the outflow portion 310 of the outer skirt 300 can extend over or transect a portion of the outflow cells 120 of the frame 102 (for example, elongated outflow cells 120 disposed at the outflow end 110 of the frame). For example, it may be desirable to extend the outer skirt 300 beyond the inflow struts 134 of the outflow cells 120 (for example, toward the outflow end 110 and closer to a mid-height of the outflow cells 120), in order to increase a surface area for PVL sealing at the implantation site. In other examples, it may be desirable to minimize the extent the skirt covers the outflow cells 120 to maximize the area of the outflow cells that can be used for coronary access (when implanted in the native aortic valve). In the illustrated example, the outflow edge 305 of the skirt terminates short of the junctions 320 formed by the intersections of the upper ends of angled struts 134 and the lower ends of axial struts 140 such that there is an axially extending gap between the outflow edge 305 and each junction 320. In some examples, the outflow edge 305 of the skirt is farther away from junctions 320 and closer to junctions 322 (formed at the intersections of struts 134 and struts 132) than is shown in FIG. 5. For example, the outflow edge 305 can be positioned halfway between the junctions 320 and the junctions 322. In other examples, the outflow edge 305 can extend to or beyond (in a direction toward the outflow end of the frame) junctions 320 such that the skirt covers the entirety of the angled struts 134. Due to the relatively large size of the outflow cells 120, the outer skirt 300 may be more susceptible to extending into the frame 102 through the outflow cells 120, for example, when the frame 102 is in a partially expanded state. The outflow leno line 304c can be elastic in some examples, as shown in FIGS. 6A-6B, to prevent the outer skirt 300 from extending into outflow cells 120 the frame 102.

In some examples, the woven yarns 316 of the outer skirt 300 can function as a barrier or curtain to prevent portions of the outer skirt 300 (for example, non-woven yarns 306 and/or texturized floating yarns 302) from extending into cells 118 of the frame 102. In some examples, as shown in FIGS. 6A-6B, the woven yarns 316 can create a barrier or curtain between the non-woven yarns 306 and the frame 102. For example, the woven yarns 316 can be disposed radially inward of the non-woven yarns 306 (FIG. 6B), such that the non-woven yarns 306 extend radially outward and away from the frame 102. The curtain or barrier created by the woven yarns 316 can prevent the non-woven yarns 306 from extending into the frame 102 through cells 118 (for example, including through outflow cells 120, etc.). The barrier created by the woven yarns 316 can extend from the inflow end 303 of the skirt 300 to the outflow end 305 of the skirt 300, such that the inflow portion 308 and the outflow portion 310 include the barrier.

In some examples, the barrier created by the woven yarns 316 can also prevent texturized portions of the outer skirt 300 (for example, texturized floating yarns 302) from extending into the frame 102. For example, non-texturized woven yarns 316 can function as a barrier or curtain between texturized floating yarns 302 (for example, texturized non-woven yarns 306 and/or texturized woven yarns 316) and the frame 102, for example, in the inflow portion 308 and/or the outflow portion 310 of the skirt 300. The non-texturized woven yarns 316 can be disposed radially inward of the texturized floating yarns 302.

In some examples, all of the floating yarns 302 of the skirt 300 can be woven yarns 316 with a first grouping of the yarns being texturized and a second grouping of the yarns being non-texturized. The second grouping of non-texturized, woven yarns can function as a barrier and can be positioned radially inwards of the texturized, woven yarns, to prevent the texturized yarns from extending radially inwards into the frame 102 through cells 118.

The outer skirt 300 can be secured to the frame 102 (or an alternate frame, as described above) by whip stitches, in-and-out stitches, or the like, that extend along a portion of the prosthetic heart valve that follows a scallop line of the leaflets and/or an outflow edge portion (at the outflow end 305) of the outer skirt 300. As one example, as shown in FIG. 5, the outer skirt 300 can be secured to struts 116 of the frame 102 that extend along the scallop line of the leaflets by whip stitches 318. In alternate examples, the outer skirt 300 can be secured at its inflow edge portion to the inflow struts 130 and at its outflow edge portion to the struts that the outflow edge portion extends across (for example, angled struts 134). In some examples, the outer skirt 300 can additionally and/or alternatively be secured to the apertures 147 in the axial struts 140 by fasteners (for example, sutures).

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a frame having an inflow end and an outflow end that is expandable between a radially compressed configuration and a radially expanded configuration; and a skirt disposed around an outer surface of the frame and having an inflow portion and an outflow portion, the skirt comprising a first plurality of axially extending fibers disposed in the inflow portion and a second plurality of axially extending fibers disposed in the outflow portion, wherein each of the first plurality of fibers include two fixed ends, wherein at least one of the second plurality of fibers includes a free end and a fixed end.

Example 2. The prosthetic heart valve of any example herein, particularly example 1, wherein the skirt further comprises a plurality of circumferentially extending floating leno lines that are spaced axially apart from one another.

Example 3. The prosthetic heart valve of any example herein, particularly example 2, wherein each fixed end is woven into one of the leno lines.

Example 4. The prosthetic heart valve of any example herein, particularly either example 2 or example 3, wherein the skirt comprises an inflow leno line, an intermediate leno line, and an outflow leno line, wherein the inflow portion is disposed between the inflow leno line and the intermediate leno line, wherein the outflow portion is disposed between the intermediate leno line and the outflow leno line.

Example 5. The prosthetic heart valve of any example herein, particularly example 4, wherein at least one of the inflow leno line, the intermediate leno line, and the outflow leno line are configured to increase in length as the frame is radially expanded from the radially compressed configuration to the radially expanded configuration.

Example 6. The prosthetic heart valve of any example herein, particularly example 5, wherein at least one of the inflow leno line, the intermediate leno line, and the outflow leno line comprise an elastic material.

Example 7. The prosthetic heart valve of any example herein, particularly any one of examples 1-6, wherein the frame comprises a plurality of interconnected struts defining a plurality of circumferentially extending rows of cells including a row of outflow cells disposed at the outflow end of the frame, wherein the row of outflow cells are longer in an axial direction relative to remaining rows of cells of the plurality of rows of cells, and wherein the skirt covers at least a portion of each outflow cell of the row of outflow cells.

Example 8. The prosthetic heart valve of any example herein, particularly any one of examples 1-7, wherein at least one fiber of the first plurality of fibers is textured.

Example 9. The prosthetic heart valve of any example herein, particularly any one of examples 1-8, wherein at least one fiber of the second plurality of fibers is textured.

Example 10. The prosthetic heart valve of any example herein, particularly any one of examples 1-9, wherein the free end is closer to the outflow end than the fixed end.

Example 11. The prosthetic heart valve of any example herein, particularly any one of examples 1-10, wherein the second plurality of fibers includes a first grouping of fixed fibers having two fixed ends and a second grouping of free fibers having a free end and a fixed end, wherein the second grouping is disposed radially outwards of the first grouping.

Example 12. A prosthetic heart valve comprising: an annular frame having an inflow end and an outflow end; and a skirt disposed around an outer surface of the frame, the skirt comprising a plurality of circumferentially-extending leno lines that are spaced axially apart from one another and a plurality of floating yarns disposed between adjacent leno lines, wherein a first grouping of the floating yarns are woven yarns that are woven into adjacent leno lines and a second grouping of the floating yarns are non-woven yarns that are not woven into at least one adjacent leno line.

Example 13. The prosthetic heart valve of any example herein, particularly example 12, wherein the plurality of leno lines comprises an inflow leno line disposed towards the inflow end, an outflow leno line disposed towards the outflow end, and an intermediate leno line disposed axially between the inflow leno line and the outflow leno line.

Example 14. The prosthetic heart valve of any example herein, particularly example 13, wherein ends of the woven yarns are catch regions that are woven into adjacent leno lines, and wherein first ends of the non-woven yarns are catch regions that are woven into adjacent leno lines and second ends of the non-woven yarns are no-catch regions that are not woven into adjacent leno lines.

Example 15. The prosthetic heart valve of any example herein, particularly example 14, wherein the catch regions of at least some of the woven yarns are woven into the inflow leno line and the intermediate leno line.

Example 16. The prosthetic heart valve of any example herein, particularly either example 14 or example 15, wherein the catch regions of at least some of the woven yarns are woven into the intermediate leno line and the outflow leno line.

Example 17. The prosthetic heart valve of any example herein, particularly any one of examples 14-16, wherein a catch region of a non-woven yarn is woven into the intermediate leno line and a no-catch region of the non-woven yarn is positioned at or adjacent to the outflow leno line.

Example 18. The prosthetic heart valve of any example herein, particularly any one of examples 13-17, wherein the outflow leno line is elastic.

Example 19. The prosthetic heart valve of any example herein, particularly example 18, wherein the frame comprises a plurality of interconnected struts defining a plurality of circumferentially extending rows of cells including a row of outflow cells disposed at the outflow end of the frame, wherein the row of outflow cells are longer in an axial direction relative to remaining rows of cells of the plurality of rows of cells, and wherein the skirt covers at least a portion of each outflow cell of the row of outflow cells.

Example 20. The prosthetic heart valve of any example herein, particularly any one of examples 12-19, wherein the non-woven yarns are disposed radially outward of the woven yarns.

Example 21. The prosthetic heart valve of any example herein, particularly any one of examples 12-20, wherein at least some of the floating yarns are texturized.

Example 22. The prosthetic heart valve of any example herein, particularly example 21, wherein all of the non-woven yarns are texturized.

Example 23. A prosthetic heart valve comprising: a frame; and a skirt coupled to the frame, the skirt comprising circumferential leno lines and axial yarns extending between the leno lines, wherein a first grouping of the axial yarns are woven into the leno lines, wherein the first grouping of the axial yarns defines a barrier between a second grouping of the axial yarns and the frame.

Example 24. The prosthetic heart valve of any example herein, particularly example 23, wherein each axial yarn of the second grouping is texturized.

Example 25. The prosthetic heart valve of any example herein, particularly either example 23 or example 24, wherein each axial yarn of the second grouping includes a free end that is not woven into one of the leno lines.

Example 26. The prosthetic heart valve of any example herein, particularly any one of examples 23-25, wherein the leno lines are axially spaced apart and define an inflow portion of the skirt and an outflow portion of the skirt.

Example 27. The prosthetic heart valve of any example herein, particularly example 26, wherein the second grouping of the axial yarns is positioned in the outflow portion of the skirt.

Example 28. The prosthetic heart valve of any example herein, particularly any one of examples 23-27, wherein at least one of the leno lines is elastically deformable.

Example 29. The prosthetic heart valve of any example herein, particularly any one of examples 23-28, wherein an outflow leno line is elastically deformable, wherein the outflow leno line is positioned at an outflow end of the skirt.

Example 30. The prosthetic heart valve of any example herein, particularly example 29, wherein the frame comprises a plurality of interconnected struts defining a plurality of circumferentially extending rows of cells including a row of outflow cells disposed at the outflow end of the frame, wherein the row of outflow cells are longer in an axial direction relative to remaining rows of cells of the plurality of rows of cells, and wherein the skirt covers at least a portion of each outflow cell of the row of outflow cells.

Example 31. The prosthetic heart valve of any example herein, particularly any one of examples 23-30, wherein the first grouping of the axial yarns are non-texturized.

Example 32. A prosthetic heart valve comprising: a frame comprising a plurality of interconnected struts defining a plurality of circumferentially extending rows of cells including a row of outflow cells disposed at an outflow end of the frame, wherein the row of outflow cells are longer in an axial direction relative to remaining rows of cells of the plurality of rows of cells; and an outer skirt coupled to the frame, the skirt comprising a plurality of circumferential yarns and a plurality of axial yarns extending between the circumferential yarns, wherein a first circumferential yarn is elastically deformable and extends across at least a portion of each outflow cell of the row of outflow cells.

Example 33. The prosthetic heart valve of any example herein, particularly example 32, wherein the circumferential yarns are woven into a leno weave.

Example 34. The prosthetic heart valve of any example herein, particularly either example 32 or example 33, wherein each axial yarn includes at least one woven region that is woven with one of the circumferential yarns.

Example 35. The prosthetic heart valve of any example herein, particularly example 34, wherein a first grouping of the axial yarns include a first woven region that is woven with the first circumferential yarn and a second woven region that is woven with a second circumferential yarn, wherein the second circumferential yarn is adjacent to the first circumferential yarn.

Example 36. The prosthetic heart valve of any example herein, particularly example 35, wherein a second grouping of the axial yarns include a woven region that is woven with the second circumferential yarn and a free region positioned adjacent to the first circumferential yarn that is not-woven into the first circumferential yarn and free to move radially outwards relative to the first circumferential yarn.

Example 37. The prosthetic heart valve of any example herein, particularly example 36, wherein the axial yarns of the second grouping are texturized.

Example 38. The prosthetic heart valve of any example herein, particularly example 35, wherein a second grouping of the axial yarns include a first woven region that is woven with the first circumferential yarn and a second woven region that is woven with the second circumferential yarn, wherein the axial yarns of the second grouping are texturized.

Example 39. The prosthetic heart valve of any example herein, particularly any one of examples 32-38, wherein each circumferential yarn is elastically deformable.

Example 40. A prosthetic heart valve comprising: a frame having an inflow end and an outflow end; and a skirt coupled to the frame, the skirt comprising a plurality of circumferential yarns and a plurality of axial yarns extending between the circumferential yarns, wherein the plurality of circumferential yarns include a first circumferential yarn that is positioned towards the outflow end and a second circumferential yarn that is positioned closer to the inflow end than the first circumferential yarn, wherein a first grouping of the axial yarns include a first woven region that is woven with the first circumferential yarn and a second woven region that is woven with the second circumferential yarn, and wherein a second grouping of the axial yarns include a woven region that is woven with the second circumferential yarn and a free region positioned adjacent to the first circumferential yarn that is not-woven into the first circumferential yarn and free to move radially outwards relative to the first circumferential yarn.

Example 41. The prosthetic heart valve of any example herein, particularly example 40, wherein axial yarns of the first grouping are non-texturized.

Example 42. The prosthetic heart valve of any example herein, particularly either example 40 or example 41, wherein axial yarns of the second grouping are texturized.

Example 43. The prosthetic heart valve of any example herein, particularly any one of examples 40-42, wherein the skirt is arranged in an alternating pattern in a circumferential direction of axial yarns of the first grouping and axial yarns of the second grouping.

Example 44. The prosthetic heart valve of any example herein, particularly any one of examples 40-43, wherein the skirt comprises a third circumferential yarn that is positioned closer to the inflow end than the second circumferential yarn, wherein a third grouping of the axial yarns include a third woven region that is woven with the third circumferential yarn and a fourth woven region that is woven with the second circumferential yarn.

Example 45. The prosthetic heart valve of any example herein, particularly example 44, wherein a fourth grouping of the axial yarns include a woven region that is woven with the third circumferential yarn and a free region positioned adjacent to the second circumferential yarn that is not-woven into the second circumferential yarn and free to move radially outwards relative to the second circumferential yarn.

Example 46. The prosthetic heart valve of any example herein, particularly example 45, wherein axial yarns of the fourth grouping are texturized.

Example 47. The prosthetic heart valve of any example herein, particularly either example 45 or example 46, wherein the skirt is arranged in an alternating pattern in a circumferential direction of axial yarns of the third grouping and axial yarns of the fourth grouping.

Example 48. The prosthetic heart valve of any example herein, particularly any one of examples 44-47, wherein axial yarns of the third grouping are non-texturized.

Example 49. The prosthetic heart valve of any example herein, particularly example 48, wherein a fifth grouping of the axial yarns include a fifth woven region that is woven with the third circumferential yarn and a sixth woven region that is woven with the second circumferential yarn, wherein axial yarns of the fifth grouping are texturized.

Example 50. The prosthetic heart valve of any example herein, particularly example 49, wherein axial yarns of the fifth grouping are disposed radially outward of axial yarns of the third grouping.

Example 51. The prosthetic heart valve of any example herein, particularly any one of examples 44-50, wherein the third circumferential yarn is positioned at an inflow end of the skirt.

Example 52. The prosthetic heart valve of any example herein, particularly any one of examples 40-51, wherein the circumferential yarns are woven in a leno weave.

Example 53. The prosthetic heart valve of any example herein, particularly any one of examples 40-52, wherein at least one of the circumferential yarns are elastic.

Example 54. The prosthetic heart valve of any example herein, particularly any one of examples 1-53, wherein the prosthetic heart valve is sterilized.

The features described herein with regard to any example can be combined with other features described in any one or more of the other examples, unless otherwise stated. For example, any one or more of the features of one prosthetic heart valve can be combined with any one or more features of another prosthetic heart valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A prosthetic heart valve comprising:
a frame having an inflow end and an outflow end that is expandable between a radially compressed configuration and a radially expanded configuration; and
a skirt disposed around an outer surface of the frame and having an inflow portion and an outflow portion, the skirt comprising a first plurality of axially extending fibers disposed in the inflow portion and a second plurality of axially extending fibers disposed in the outflow portion, wherein each of the first plurality of fibers comprises two fixed ends, wherein at least one of the second plurality of fibers comprises a free end and a fixed end.

2. The prosthetic heart valve of claim 1, wherein the skirt further comprises a plurality of circumferentially extending leno lines that are spaced axially apart from one another.

3. The prosthetic heart valve of claim 2, wherein each fixed end is woven into one of the leno lines.

4. The prosthetic heart valve of claim 2, wherein the skirt comprises an inflow leno line, an intermediate leno line, and an outflow leno line, wherein the inflow portion is disposed between the inflow leno line and the intermediate leno line, wherein the outflow portion is disposed between the intermediate leno line and the outflow leno line.

5. The prosthetic heart valve of claim 4, wherein at least one of the inflow leno line, the intermediate leno line, and the outflow leno line are configured to increase in length as the frame is radially expanded from the radially compressed configuration to the radially expanded configuration.

6. The prosthetic heart valve of claim 5, wherein at least one of the inflow leno line, the intermediate leno line, and the outflow leno line comprise an elastic material.

7. The prosthetic heart valve of claim 1, wherein at least one fiber of the first plurality of fibers is textured.

8. The prosthetic heart valve of claim 1, wherein at least one fiber of the second plurality of fibers is textured.

9. The prosthetic heart valve of claim 1, wherein the free end is closer to the outflow end than the fixed end.

10. A prosthetic heart valve comprising:
an annular frame having an inflow end and an outflow end; and
a skirt disposed around an outer surface of the frame, the skirt comprising a plurality of circumferentially-extending leno lines that are spaced axially apart from one another and a plurality of floating yarns disposed between adjacent leno lines, wherein a first grouping of the floating yarns are woven yarns that are woven into adjacent leno lines and a second grouping of the floating yarns are non-woven yarns that are not woven into at least one adjacent leno line.

11. The prosthetic heart valve of claim 10, wherein the plurality of leno lines comprises an inflow leno line disposed towards the inflow end, an outflow leno line disposed towards the outflow end, and an intermediate leno line disposed axially between the inflow leno line and the outflow leno line.

12. The prosthetic heart valve of claim 11, wherein ends of the woven yarns are catch regions that are woven into adjacent leno lines, and wherein first ends of the non-woven yarns are catch regions that are woven into adjacent leno lines and second ends of the non-woven yarns are no-catch regions that are not woven into adjacent leno lines.

13. The prosthetic heart valve of claim 12, wherein the catch regions of at least some of the woven yarns are woven into the inflow leno line and the intermediate leno line.

14. The prosthetic heart valve of claim 12, wherein the catch regions of at least some of the woven yarns are woven into the intermediate leno line and the outflow leno line.

15. The prosthetic heart valve of claim 12, wherein a catch region of a non-woven yarn is woven into the intermediate leno line and a no-catch region of the non-woven yarn is positioned at or adjacent to the outflow leno line.

16. The prosthetic heart valve of claim 10, wherein the frame comprises a plurality of interconnected struts defining a plurality of circumferentially extending rows of cells including a row of outflow cells disposed at the outflow end of the frame, wherein the row of outflow cells are longer in an axial direction relative to remaining rows of cells of the plurality of rows of cells, and wherein the skirt covers at least a portion of each outflow cell of the row of outflow cells.

17. The prosthetic heart valve of claim 10, wherein at least some of the floating yarns are texturized.

18. A prosthetic heart valve comprising:
a frame; and
a skirt coupled to the frame, the skirt comprising circumferential leno lines and axial yarns extending between the leno lines, wherein a first grouping of the axial yarns are woven into the leno lines, wherein the first grouping of the axial yarns defines a barrier between a second grouping of the axial yarns and the frame.

19. The prosthetic heart valve of claim 18, wherein each axial yarn of the second grouping includes a free end that is not woven into one of the leno lines.

20. The prosthetic heart valve of claim 18, wherein at least one of the leno lines is elastically deformable.
